(19) United States
(12) Patent Application Publication
(10) Pub. No.: US 2025/0248618 A1
(43) Pub. Date: Aug. 7, 2025

(54) MAGNETIC RESONANCE SYSTEM AND METHOD FOR MEASURING REGIONAL BODY FAT CONTENT USING SAME

(71) Applicant: WUXI MARVEL STONE HEALTHCARE CO., LTD., Wuxi (CN)

(72) Inventors: Yue ZHAO, Chengdu (CN); Hai LUO, Chengdu (CN); Chao WANG, Chengdu (CN); Yunhao XIE, Chengdu (CN); Jianxiong HU, Chengdu (CN); Wenkui HOU, Chengdu (CN); Xiao CHEN, Chengdu (CN); Min WU, Chengdu (CN); Ziyue WU, Chengdu (CN)

(73) Assignee: WUXI MARVEL STONE HEALTHCARE CO., LTD., Wuxi, JS (CN)

(21) Appl. No.: 19/091,941

(22) Filed: Mar. 27, 2025

Related U.S. Application Data
(63) Continuation-in-part of application No. PCT/CN2022/130125, filed on Nov. 4, 2022.

(30) Foreign Application Priority Data
Oct. 9, 2022 (CN) 202211230518.5

(51) Int. Cl. A61B 5/055; A61B 5/00; G01R 33/3415; G01R 33/36

(52) U.S. Cl. CPC A61B 5/055; A61B 5/4872; G01R 33/3415; G01R 33/3607

(57) ABSTRACT

The provided is a magnetic resonance (MR) system and a method for measuring a regional body fat content using same. The method includes: when an object to be measured is a single-substance object: acquiring, by a corresponding radio frequency (RF) pulse sequence, an MR signal of the object to be measured; processing the acquired MR signal, and obtaining a target characteristic parameter value; calibrating, by phantoms with known different fat contents, the characteristic parameter, and establishing a correspondence between the characteristic parameter and the fat contents; and determining a fat content corresponding to the target characteristic parameter value (S1); and when the object to be measured is a mixed-substance object including a fat component and a non-fat component: acquiring an MR signal of the object to be measured; determining undetermined coefficients of the fat component and the non-fat component; and calculating a fat content of the object to be measured.

### CROSS-REFERENCE TO THE RELATED APPLICATIONS

This application is a continuation-in-part application of International Application No. PCT/CN2022/130125, filed on Nov. 4, 2022, which is based upon and claims priority to Chinese Patent Application No. 202211230518.5, filed on Oct. 9, 2022, the entire contents of which are incorporated herein by reference.

### TECHNICAL FIELD

The present disclosure belongs to the technical field of magnetic resonance (MR), and in particular relates to an MR system and a method for measuring a regional body fat content using the same.

### BACKGROUND

In MR system testing, the regional body fat content of subjects varies due to their different body shapes. Fat, along with proteins and carbohydrates, is one of the three essential components of the human body. In specific test scenarios, regional body fat content can seriously affect the test results. Therefore, precise determination of the regional body fat content is of great significance for the application of MR system testing.

In the prior art, the method for measuring regional body fat content using the MR system first scans the subcutaneous area, followed by the measurement on the scanned image through MR image analysis software. However, this method requires scanning before measurement, resulting in high measurement cost, and it relies on professional MR image analysis software, leading to operational complexity.

### SUMMARY

An objective of the present disclosure is to provide an MR system and a method for measuring a regional body fat content using same. The present disclosure solves the technical problems in the prior art. That is, the measurement method requires scanning before measurement, resulting in high measurement cost, and it relies on professional MR image analysis software, leading to operational complexity.

In order to achieve the above objective, the present disclosure adopts the following technical solutions:

A first aspect provides an MR system for measuring a regional body fat content, including: a data processing subsystem, a radio frequency (RF) subsystem, and a magnet device, where
the RF subsystem includes a spectrum analyzer, a power amplifier, a preamplifier, a transmit-receive (T/R) switch, and a surface coil module; the surface coil module includes at least one set of surface coils; and a depth of an excitation area of the at least one set of surface coils is adapted to a subcutaneous depth.

In a possible design, the surface coil module includes a set of TR integrated surface coils; and a depth of an excitation area and a depth of a receiving area of the surface coils are both adapted to the subcutaneous depth.

In a possible design, the surface coil module includes a set of surface excitation coils and a set of surface receiving coils; and a depth of an excitation area of the surface excitation coils is adapted to the subcutaneous depth.

In a possible design, the set of surface excitation coils includes a plurality of surface excitation coils.

A second aspect provides a method for measuring a regional body fat content using the MR system, including:
when an object to be measured is regarded as a single-substance object: acquiring, by a corresponding RF pulse sequence, an MR signal of the object to be measured according to a characteristic parameter to be measured; processing the acquired MR signal, and obtaining a target characteristic parameter value corresponding to the object to be measured; calibrating, by phantoms with known different fat contents, the characteristic parameter, and establishing a correspondence between the characteristic parameter to be measured and the fat contents; and determining a fat content corresponding to the target characteristic parameter value according to the correspondence, where the characteristic parameter at least includes a longitudinal relaxation time T1, a transverse relaxation time T2, and/or an apparent diffusion coefficient (ADC) D; and
when the object to be measured is regarded as a mixed-substance object including a fat component and a non-fat component: acquiring, by a corresponding RF pulse sequence, an MR signal of the object to be measured, where the MR signal includes an MR signal of the fat component and an MR signal of the non-fat component; determining, by a multi-parameter fitting-based numerical calculation method, undetermined coefficients of the fat component and the non-fat component in the MR signal, respectively; and calculating a fat content of the object to be measured according to the undetermined coefficients.

In a possible design, the method includes: when the object to be measured is regarded as a single-substance object:
when the characteristic parameter to be measured is the longitudinal relaxation time T1, acquiring, by a varied-repetition time-Carr-Purcell-Meiboom-Gill (VTR-CPMG) sequence, the MR signal of the object to be measured;
when the characteristic parameter to be measured is the transverse relaxation time T2, acquiring, by a Carr-Purcell-Meiboom-Gill (CPMG) sequence, the MR signal of the object to be measured; and
when the characteristic parameter to be measured is the ADC D, acquiring, by a spin echo (SE)-Diffusion sequence, the MR signal of the object to be measured.

In a possible design, the processing the acquired MR signal, and obtaining a target characteristic parameter value corresponding to the object to be measured includes:
averaging MR signals acquired by the VTR-CPMG sequence based on a series of different repetition times (TR), and obtaining a signal intensity value A1 corresponding to different recovery times of a longitudinal magnetization vector as follows:

$$A 1 = {A}_{01} ( a - {be}^{( - TR / T 1 )} {e}^{( - TE / T 2 )} ) \qquad ( 1 )$$

where, A₀₁ is a signal intensity corresponding to a maximum longitudinal magnetization vector; a and b are function parameters used to describe a recovery value of the longitudinal magnetization vector along a direction of a main magnetic field B₀, and in a case of ideal saturation-recovery, a=b=1; e is a natural logarithm; and TE is a fixed echo time (TE); and
calculating the longitudinal relaxation time T1 based on a minimum function min constraint as follows:

$$min \{ ‖ {S}_{1} - {\sum }_{i = 1}^{N} A {1}_{i} ( {a}_{i} - {b}_{i} {e}^{( - TR / T {1}_{i} )} {e}^{( - TE / T 2 )} ) ‖ \} \qquad ( 2 )$$

where, ∥ ∥ is a 2-norm of the vector; S₁ is an intensity of the acquired VTR-CPMG echo signal; N is a total number of substances in the object to be measured for which the longitudinal relaxation time T1 needs to be estimated; A1ᵢ is a total signal intensity of an i^{th} substance; aᵢ and bᵢ are function parameters used for the i^{th} substance to describe the recovery value of the longitudinal magnetization vector along the direction of the main magnetic field B₀; and T1ᵢ is the longitudinal relaxation time T1 of the i^{th} substance.

In a possible design, the processing the acquired MR signal, and obtaining a target characteristic parameter value corresponding to the object to be measured includes: averaging CPMG signals with same echo spacing acquired a plurality of times based
on a fixed TE, and obtaining a decay signal intensity value A₂ of a transverse magnetization vector based on the fixed TE as follows:

$$A 2 = {A}_{02} {e}^{( - t 1 / {T}_{2} )} \qquad ( 3 )$$

where, A₀₂ is a signal intensity corresponding to a maximum transverse magnetization vector; e is a natural logarithm; and t1 is a TE; and
subjecting the CPMG echo signals to single-exponential fitting, and calculating, by a minimum function min constraint, the transverse relaxation time T2 as follows:

$$min \{ ‖ {S}_{2} - {\sum }_{i = 1}^{N} A {2}_{i} {e}^{- t 2 / T {2}_{i} )} ‖ \} \qquad ( 4 )$$

where, S₂ is an intensity of the acquired CPMG echo signal; A2ᵢ is a signal amplitude of an i^{th} substance; N is a total number of substances in the object to be measured; t2 is a time calculated from a maximum transverse magnetization intensity; and T₂ᵢ is the transverse relaxation time T2 of the i^{th} substance.

In a possible design, the processing the acquired MR signal, and obtaining a target characteristic parameter value corresponding to the object to be measured includes:
performing phase correction and accumulation on an acquired SE-Diffusion signal, and obtaining a correspondence between a signal intensity and a diffusion encoding time as follows:

$${S}_{3} = A 3 {e}^{- \frac{- {γ}^{2} {G}^{2} {tEE}^{3} D}{12} - \frac{tEE}{T 2}} \qquad ( 5 )$$

where, A3 is an amplitude of the SE-Diffusion signal; γ is a magnetogyric ratio; G is a strength of a gradient magnetic field; and tEE is the diffusion encoding time; and
changing the diffusion encoding time tEE, averaging a series of acquired SE-Diffusion signals with different degrees of diffusion weighting, and calculating, by a minimum function min constraint, the ADC D as follows:

$$min \{ ‖ {S}_{3} - {\sum }_{i = 1}^{N} A {3}_{i} {e}^{- \frac{- {γ}^{2} {G}^{2} {tEE}^{3} Di}{12} - \frac{tEE}{T 2}} ‖ \} \qquad ( 6 )$$

where, S₃ is an intensity of the acquired SE-Diffusion signal; N is a total number of substances in the object to be measured for which the apparent diffusion coefficients D need to be estimated; and A3ᵢ is a total signal intensity of an i^{th} substance.

In a possible design, the calibrating, by phantoms with known different fat contents, the characteristic parameter, and establishing a correspondence between the characteristic parameter to be measured and the fat contents includes:
mixing the phantoms with known different fat contents with a same proton density fat fraction (PDFF) phantom, acquiring reconstruction data of a mixed signal based on a corresponding RF pulse sequence, and obtaining a corresponding characteristic parameter; and
establishing the correspondence between the characteristic parameter and the regional body fat content based on the reconstruction data of the mixed signal of a preset sample size and the corresponding characteristic parameter.

In a possible design, the method includes: when the object to be measured is regarded as a mixed-substance object: acquiring, by a VTR-CPMG sequence, a CPMG sequence, or a SE-Diffusion sequence, the MR signal of the object to be measured.

In a possible design, when acquiring, by the VTR-CPMG sequence, the MR signal of the object to be measured, determining, by the multi-parameter fitting-based numerical calculation method, the undetermined coefficients of the fat component and the non-fat component in the MR signal, respectively; and calculating the fat content of the object to be measured according to the undetermined coefficients includes:
establishing an MR signal calculation equation as follows:

$$min \{ ‖ {S}_{1} - {B}_{1} ( {a}_{1} - {b}_{1} {e}^{( - T R / T {1}_{1} )} {e}^{( - T E / T 2 )} ) - {B}_{2} ( {a}_{2} - {b}_{2} {e}^{( - T R / T {1}_{2} )} {e}^{( - T E / T 2 )} ) ‖ \} \qquad ( 7 )$$

where, S₁ is an intensity of an acquired VTR-CPMG echo signal; B₁ is a total signal intensity of a substance component of the non-fat component; and B₂ is a total signal intensity of a substance component of the fat component; and
determining, by the multi-parameter fitting-based numerical calculation method, the undetermined coefficients B₁ and B₂; and calculating a PDFF of the object to be measured according to the undetermined coefficients as follows:

$$PDFF = \frac{{B}_{2}}{{B}_{1} + {B}_{2}} \times 1 \; 00 \% . \qquad ( 8 )$$

In a possible design, when acquiring, by the CPMG sequence, the MR signal of the object to be measured, determining, by the multi-parameter fitting-based numerical calculation method, the undetermined coefficients of the fat component and the non-fat component in the MR signal, respectively; and calculating the fat content of the object to be measured according to the undetermined coefficients includes:
establishing an MR signal calculation equation as follows:

$$min \{ ‖ {S}_{2} - {B}_{1} {e}^{- t 2 / T {2}_{1}} - {B}_{2} {e}^{- t 2 / T {2}_{2}} ‖ \} \qquad ( 9 )$$

where, S₂ is an intensity of an acquired CPMG echo signal; B₁ is a total signal intensity of a substance component of the non-fat component; and B₂ is a total signal intensity of a substance component of the fat component; and
determining, by the multi-parameter fitting-based numerical calculation method, the undetermined coefficients B₁ and B₂; and calculating a PDFF of the object to be measured according to the undetermined coefficients as follows:

$$PDFF = \frac{{B}_{2}}{{B}_{1} + {B}_{2}} \times 1 \; 00 \% . \qquad ( 10 )$$

In a possible design, when acquiring, by the SE-Diffusion sequence, the MR signal of the object to be measured, determining, by the multi-parameter fitting-based numerical calculation method, the undetermined coefficients of the fat component and the non-fat component in the MR signal, respectively; and calculating the fat content of the object to be measured according to the undetermined coefficients includes:
establishing an MR signal calculation equation as follows:

$$min \{ ‖ {S}_{3} - {B}_{1} {e}^{- \frac{- {γ}^{2} {G}^{2} t E {E}^{3} D 1}{12} - \frac{tEE}{T 2}} - {B}_{2} {e}^{- \frac{- {γ}^{2} {G}^{2} t E {E}^{3} D 2}{12} - \frac{tEE}{T 2}} ‖ \} \qquad ( 11 )$$

determining, by the multi-parameter fitting-based numerical calculation method, undetermined coefficients B₁ and B₂; and calculating a PDFF of the object to be measured according to the undetermined coefficients as follows:

$$PDFF = \frac{{B}_{2}}{{B}_{1} + {B}_{2}} \times 1 \; 00 \% . \qquad ( 12 )$$

In a possible design, each time when the characteristic parameter is calibrated by the phantoms with known different fat contents, phantoms with different fat contents and a same PDFF phantom are placed on the surface coil module of the MR system.

In a third aspect, the present disclosure provides a computer device, including a memory, a processor, and a transceiver which are sequentially connected in communication, where the memory is configured to store a computer program; the transceiver is configured to send and receive messages; and the processor is configured to read the computer program and execute the method for measuring a regional body fat content using the MR system described in any possible design of the first aspect.

In a fourth aspect, the present disclosure provides a computer-readable storage medium, where the computer-readable storage medium is configured to store an instruction, where the instruction is run on a computer to execute the method for measuring a regional body fat content using the MR system described in any possible design of the first aspect.

In a fifth aspect, the present disclosure provides a computer program product including an instruction, where when the instruction is run on a computer, the computer executes the method for measuring a regional body fat content using the MR system as described in any possible design of the first aspect.

Compared with the prior art, the present disclosure has the following beneficial effects:
1. The MR system of the present disclosure can acquire only the MR signals of the body surface as much as possible by adapting the depth of the surface excitation area of the surface coil module to the subcutaneous depth, so as to provide hardware support for the subsequent measurement of regional body fat content.
2. In the method for measuring a regional body fat content using the MR system in the present disclosure, when the object to be measured is regarded as a single-substance object, the MR signal of the object to be measured is acquired by a corresponding RF pulse sequence according to a characteristic parameter to be measured. The acquired MR signal is processed, and a target characteristic parameter value corresponding to the object to be measured is obtained. The characteristic parameter is calibrated by phantoms with known different fat contents, and a correspondence between the characteristic parameter to be measured and the fat contents is established. A fat content corresponding to the target characteristic parameter value is determined according to the correspondence, where the characteristic parameter at least includes a longitudinal relaxation time T1, a transverse relaxation time T2, and/or an ADC D. When the object to be measured is regarded as a mixed-substance object including a fat component and a non-fat component, the MR signal of the object to be measured is acquired by a corresponding RF pulse sequence. Undetermined coefficients of the fat component and the non-fat component in the MR signal are respectively determined by a multi-parameter fitting-based numerical calculation method. The fat content of the object to be measured is calculated according to the undetermined coefficients. Therefore, the present disclosure provides a calibration method for the fat content of a single-substance object, and provides a parameter fitting calculation method for the fat content of a mixed-substance object. The measurement results are accurate, providing solid data support for the testing of the MR system.

### BRIEF DESCRIPTION OF THE DRAWINGS

FIG. 1 is a structural block diagram of an MR system in an embodiment of the present disclosure;

FIG. 2 is a structural schematic diagram of a surface coil in the embodiment of the present disclosure;

FIG. 3 is a structural schematic diagram of another surface coil in the embodiment of the present disclosure;

FIG. 4 is a structural schematic diagram of yet another surface coil in the embodiment of the present disclosure;

FIG. 5 is a flowchart of a method for measuring a regional body fat content using the MR system in the embodiment of the present disclosure;

FIG. 6 is a schematic diagram of a VTR-CPMG sequence in the embodiment of the present disclosure;

FIG. 7 is a schematic diagram of a CPMG sequence in the embodiment of the present disclosure;

FIG. 8 is a schematic diagram of a SE-Diffusion sequence in the embodiment of the present disclosure; and

FIG. 9 is a schematic diagram of placement positions of phantoms with different fat contents and a PDFF phantom in the embodiment of the present disclosure.

### DETAILED DESCRIPTION OF THE EMBODIMENTS

In order to more clearly illustrate the technical solutions in the embodiments of the present disclosure or the prior art, the present disclosure will be briefly introduced below in combination with the drawings and the description of the embodiments or the prior art. Obviously, the following description of the structure of the drawings is only some embodiments of the present disclosure. For ordinary technicians in this field, other drawings can also be obtained according to these drawings without creative work. It should be noted here that the description of these embodiments is used to help understand the present disclosure, but does not constitute a limitation of the present disclosure.

### Embodiment

The present disclosure aims to solve the technical problems in the prior art. That is, the measurement method requires scanning before measurement, resulting in high measurement cost, and it relies on professional MR image analysis software, leading to operational complexity. To this end, the embodiment of the present disclosure provides a method for measuring a regional body fat content using an MR system. The present disclosure provides a calibration method for the fat content of a single-substance object, and provides a parameter fitting calculation method for the fat content of a mixed-substance object. The measurement results are accurate, providing solid data support for MR system testing. The testing of the MR system can be applied to test indicators of health problems caused by excessively high or low fat content, such as in a testing system for fatty liver.

First, on the one hand, the embodiment of the present disclosure provides an MR system for sampling and processing an MR signal of an object to be measured, where the object to be measured is preferably a human body. Specifically, the MR system includes: a data processing subsystem, an RF subsystem, and a magnet device. The data processing subsystem is connected to the RF subsystem, and the magnet device is located at one side of the RF subsystem, for providing a magnetic field required by the entire MR system, that is, generating static magnetic field B₀.

As shown in FIG. 1, the RF subsystem includes a spectrum analyzer, a power amplifier, a preamplifier, a T/R switch, and a surface coil module. The surface coil module includes at least one surface coils. A depth of an excitation area of the at least one surface coils is adapted to a subcutaneous depth, that is, the depth of excitation area of the surface coil is set to be shallow, adapted to the subcutaneous depth, so as to acquire only the MR signals of the body surface as much as possible. Preferably, the depth of the excitation area of the surface coil can be set to 3-5 cm. For example, if the depth of the subcutaneous surface is 3 cm, the depth of the excitation area of the surface coil can be set to about 3 cm. The spectrum analyzer is separately connected to the data processing subsystem, the preamplifier, the power amplifier, and the T/R switch. The preamplifier and the power amplifier are separately connected to the T/R switch. The T/R switch is connected to the surface coil module.

It should be noted that the surface coil module is configured to generate a B₁ field perpendicular to the static magnetic field B₀ so as to excite nuclei in a fat area of the body surface and also to receive an MR echo signal. Specifically, the surface coil includes but is not limited to the following structural forms:

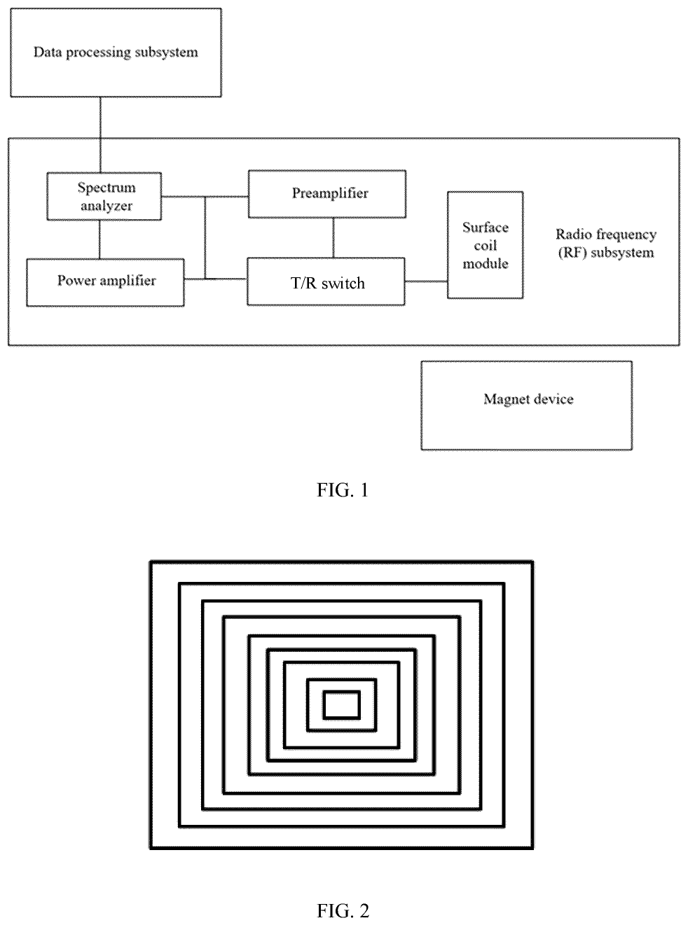

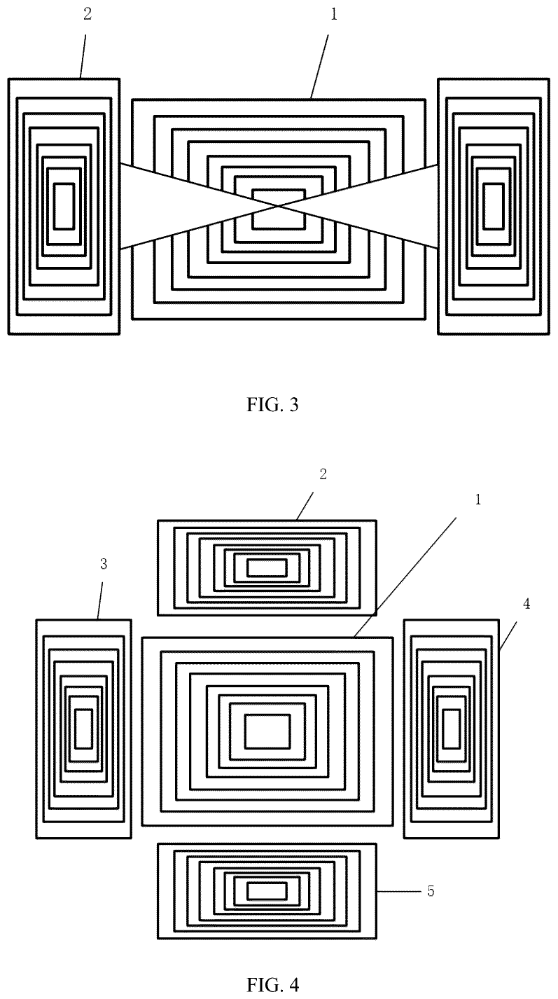

As shown in FIG. 2, in a specific implementation, the surface coil module includes a set of TR integrated surface coils, and the depth of the excitation area and a depth of a receiving area of the surface coils are both adapted to the subcutaneous depth. Specifically, the excitation coil and the receiving coil of the surface coil module are set to the same coil, which can realize both the transmission of RF pulses and the reception of MR signals.

As shown in FIG. 3, in a specific implementation, the surface coil module includes a set of surface excitation coils and a set of surface receiving coils, and the depth of the excitation area of the surface excitation coils is adapted to the subcutaneous depth. Specifically, the surface coil module includes two sets of coils, namely a set of surface excitation coils for RF pulse transmission (the surface excitation area is relatively shallow to adapt to the subcutaneous depth) and a set of receiving coils for receiving the MR echo signal (the area of the receiving signal can be relatively shallow or relatively deep, which is not limited here). In the figure, 1 denotes the receiving coil, and 2 denotes the surface excitation coil with two ends wound with coils.

As shown in FIG. 4, in a specific implementation, based on the coil shown in FIG. 3, a set of surface excitation coils includes a plurality of surface excitation coils. Specifically, the surface coil module includes two sets of coils, namely a set of surface excitation coils for RF pulse transmission, including a plurality of coils (the surface excitation area is relatively shallow to adapt to the subcutaneous depth), and a set of receiving coils for receiving the MR echo signals. There can be one or more receiving coils (the area of the receiving signal can be relatively shallow or relatively deep, which is not limited here). In FIG. 4, 1 denotes the receiving coil, and 2, 3, 4, and 5 denote the surface excitation coils each including four coils.

Of course, it can be understood that the structural form and number of the surface coils in the embodiment of the present disclosure are not limited to the above examples. The surface coils can be arranged in a planar combination or a spatial combination. Specifically, the layout can be set correspondingly according to the coverage range of the target area of the body surface for acquisition, which is not limited here.

Based on the above disclosed content, the embodiment of the present disclosure can acquire only the MR signals of the body surface as much as possible by adapting the depth of the surface excitation area of the surface coil module to the subcutaneous depth, so as to provide hardware support for the subsequent measurement of the regional body fat content.

The method for measuring a regional body fat content using the MR system provided in the embodiment of the present disclosure will be described in detail below.

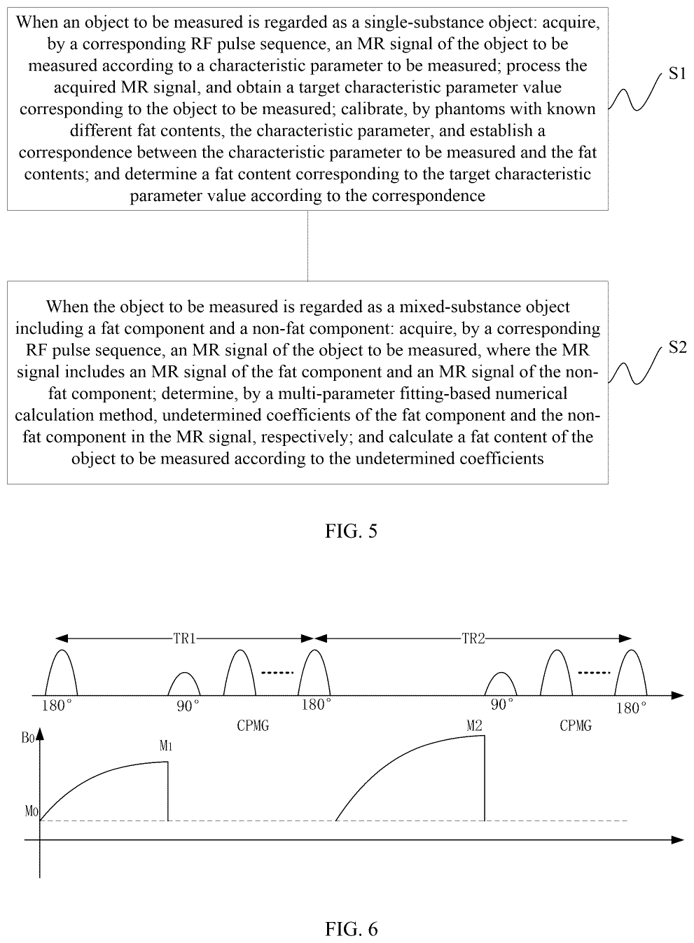

As shown in FIGS. 5 to 9, a second aspect of the embodiment of the present disclosure provides a method for measuring a regional body fat content using the MR system. The method includes but is not limited to steps S1 to S4.
Step S1. When an object to be measured is regarded as a single-substance object, an MR signal of the object to be measured is acquired by a corresponding RF pulse sequence according to a characteristic parameter to be measured. The acquired MR signal is processed, and a target characteristic parameter value corresponding to the object to be measured is obtained. The characteristic parameter is calibrated by phantoms with known different fat contents, and a correspondence between the characteristic parameter to be measured and the fat contents is established. A fat content corresponding to the target characteristic parameter value is determined according to the correspondence, where the characteristic parameter at least includes longitudinal relaxation time T1, transverse relaxation time T2, and/or apparent diffusion coefficient (ADC) D.

It should be noted that the acquisition of MR signals requires the design of an RF pulse sequence with a certain bandwidth and amplitude. Different RF pulse sequences are designed according to the characteristic parameters required for measurement, which are emitted by the excitation coil of the surface coil module. An MR signal is generated within the excitation range of the surface coil, and received by the receiving coil of the surface coil module. Preferably, the characteristic parameter required for MR measurement in the embodiment of the present disclosure includes one or a combination of more of the following parameters.

First, longitudinal relaxation time T1, which refers to a time constant for a rate characteristic by which the longitudinal magnetization vector of the substance along the direction of the main magnetic field B₀ recovers to an initial magnetization vector. As shown in FIG. 6, when the characteristic parameter to be measured is the transverse relaxation time T2, according to the principle of a partial saturation recovery pulse sequence, a Carr-Purcell-Meiboom-Gill (CPMG) sequence is used to acquire the MR signal of the object to be measured. A varied-repetition time-Carr-Purcell-Meiboom-Gill (VTR-CPMG) sequence is a sequence based on a standard CPMG sequence which sets different repetition times (TR) for scanning. In a schematic diagram of the VTR-CPMG sequence, the TR of two consecutive CPMG sequences varies, leading to different maximum recovery values of the longitudinal magnetization vector M₁ along the direction of the main magnetic field B₀.

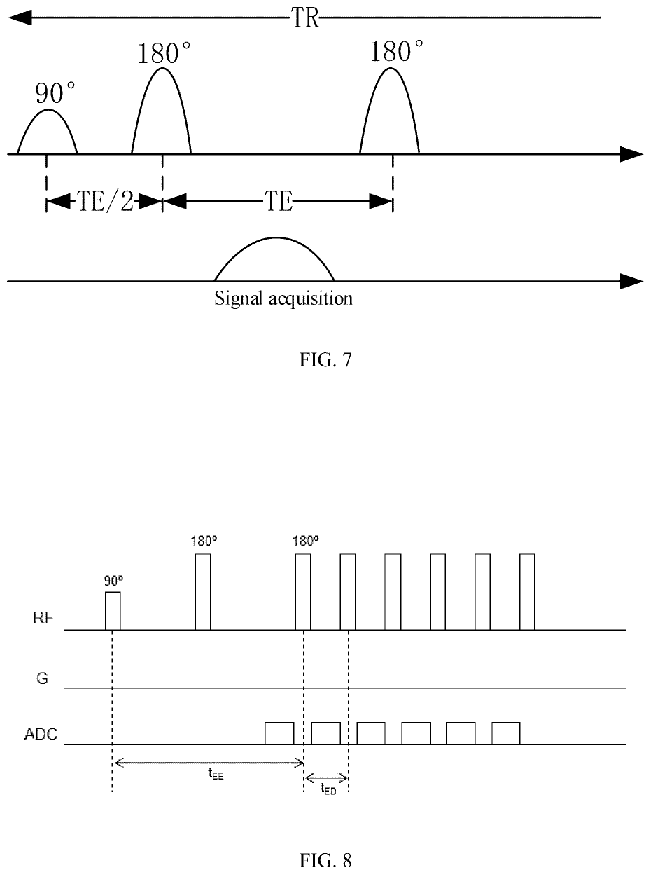

Second, transverse relaxation time T2, which refers to a time constant for the decay rate characteristic of the transverse magnetization vector M_{xy} component. When the characteristic parameter to be measured is the transverse relaxation time T2, the CPMG sequence is used to acquire the MR signal of the object to be measured. As shown in FIG. 7, the CPMG sequence using hard pulses is an echo pulse sequence derived from a spin echo (SE) pulse sequence, where a plurality of 180° pulses are applied to obtain a plurality of echo signals. FIG. 7 only shows two 180° pulses. After a 90° pulse, a dephasing period of 0.5 TE occurs. Subsequently, a 180° refocusing pulse is applied, generating a first echo signal at t=TE. Then another dephasing period comes. At t=1.5TE, a second 180° refocusing pulse is applied, and similarly, at t=2TE, the transverse magnetization vector rephases to form a second echo signal. This process is repeated to generate a plurality of echo signals. The amplitude of the echo signal gradually decreases due to the spin-spin relaxation effect.

Third, apparent diffusion coefficient (ADC) D. Diffusion refers to a type of random motion of molecules in a medium, which is called thermal motion or Brownian motion of molecules. The diffusion performance of different tissues or similar tissues under different physiological and pathological conditions is very different, so diffusion is a very important type of tissue information. In nuclear magnetic resonance (NMR), in order to detect diffusion information, a very strong gradient magnetic field is generally applied after the signal is excited. Due to the existence of molecular diffusion, molecular dephasing occurs in the voxel, thereby causing signal decay. The different degrees of signal decay represent the strength of the diffusion motion, which is generally characterized by the ADC. As shown in FIG. 8, when the characteristic parameter to be measured is the ADC D, the SE-Diffusion sequence is used to acquire the MR signal of the object to be measured. After the 90° excitation pulse, a natural gradient magnetic field diffusely encodes the signal at the encoding time of tEE. A SE signal can be acquired at the TE point after the first 180° pulse. In order to improve the testing sensitivity, after the first SE, a plurality of 180° pulses are continuously applied to repeatedly rephase the transverse magnetization and acquire the corresponding echo signals.

Of course, it can be understood that the characteristic parameters in the embodiment of the present disclosure are not limited to the above examples, and any other parameters that can reflect the characteristics of the body surface MR echo signal are within the protection scope of the present disclosure, and will not be repeated here.

It should be noted that the acquired MR signal is processed in the data processing subsystem, and different signal processing methods are used according to different acquisition sequences so as to obtain the reflected characteristic parameters, specifically as follows.

In a specific implementation of the step S1, the acquired MR signal is processed to obtain the target characteristic parameter value corresponding to the object to be measured as follows.

MR signals acquired by the VTR-CPMG sequence based on a series of different TR are averaged, and signal intensity value A1 corresponding to different recovery times of a longitudinal magnetization vector is obtained as follows:

$$A 1 = {A}_{01} ( a - b {e}^{( - T R / T 1 )} {e}^{( - T E / T 2 )} ) \qquad ( 1 )$$

where, A₀₁ is a signal intensity corresponding to a maximum longitudinal magnetization vector; a and b are function parameters used to describe a recovery value of the longitudinal magnetization vector along a direction of main magnetic field B₀, and in a case of ideal saturation-recovery, a=b=1; e is a natural logarithm; and TE is a fixed TE.

The longitudinal relaxation time T1 is calculated based on a minimum function min constraint as follows:

$$min \{ ‖ {S}_{1} - {\sum }_{i = 1}^{N} A {1}_{i} ( {a}_{i} - {b}_{i} {e}^{( - T R / T {1}_{i} )} {e}^{( - T E / T 2 )} ) ‖ \} \qquad ( 2 )$$

where, ∥ ∥ is a 2-norm of the vector; S₁ is an intensity of the acquired VTR-CPMG echo signal; NV is a total number of substances in the object to be measured for which the longitudinal relaxation time T1 needs to be estimated; A1ᵢ is a total signal intensity of an i^{th} substance; aᵢ and bᵢ are function parameters used for the i^{th} substance to describe the recovery value of the longitudinal magnetization vector along the direction of the main magnetic field B₀; and T1ᵢ is the longitudinal relaxation time T1 of the i^{th} substance.

In another specific implementation of the step S1, the acquired MR signal is processed to obtain the target characteristic parameter value corresponding to the object to be measured as follows.

CPMG signals with same echo spacing acquired a plurality of times based on a fixed TE are averaged, and decay signal intensity value A₂ of a transverse magnetization vector based on the fixed TE is obtained as follows:

$$A 2 = {A}_{02} {e}^{( - t 1 / {T}_{2} )} \qquad ( 3 )$$

where, A₀₂ is a signal intensity corresponding to a maximum transverse magnetization vector; e is a natural logarithm; and t1 is a TE.

The CPMG echo signals are subjected to single-exponential fitting, and the transverse relaxation time T2 is calculated by a minimum function min constraint as follows:

$$min \{ ‖ {S}_{2} - {\sum }_{i = 1}^{N} A {2}_{i} {e}^{- t 2 / T {2}_{i} )} ‖ \} \qquad ( 4 )$$

where, S₂ is an intensity of the acquired CPMG echo signal; A2ᵢ is a signal amplitude of an i^{th} substance; N is a total number of substances in the object to be measured; t2 is a time calculated from a maximum transverse magnetization intensity; and T₂ᵢ is the transverse relaxation time T2 of the i^{th} substance.

In another specific implementation of the step S1, the acquired MR signal is processed to obtain the target characteristic parameter value corresponding to the object to be measured as follows.

Phase correction and accumulation are performed on an acquired SE-Diffusion signal, and a correspondence between a signal intensity and a diffusion encoding time is obtained as follows:

$${S}_{3} = A 3 {e}^{- \frac{- {γ}^{2} {G}^{2} t E {E}^{3} D}{12} - \frac{tEE}{T 2}} \qquad ( 5 )$$

where, A3 is an amplitude of the SE-Diffusion signal; γ is a magnetogyric ratio; G is a strength of a gradient magnetic field; and tEE is the diffusion encoding time.

The diffusion encoding time tEE is changed, a series of acquired SE-Diffusion signals with different degrees of diffusion weighting are averaged, and the ADC D is calculated by a minimum function min constraint as follows:

$$min \{ ‖ {S}_{3} - {\sum }_{i = 1}^{N} A {3}_{i} {e}^{- \frac{- {γ}^{2} {G}^{2} t E {E}^{3} Di}{12} - \frac{tEE}{T 2}} ‖ \} \qquad ( 6 )$$

where, S₃ is an intensity of the acquired SE-Diffusion signal; N is a total number of substances in the object to be measured for which the apparent diffusion coefficients D need to be estimated; and A3ᵢ is a total signal intensity of an i^{th} substance.

It should be noted that when the object to be measured is a single-substance object with only one substance, the longitudinal relaxation time T1, the transverse relaxation time T2, and/or the ADC D of the test target can be estimated through a local optimal solution of nonlinear programming, but the fat content cannot be directly obtained, so fat content calibration is required.

In a specific implementation of the step S1, the characteristic parameter is calibrated by phantoms with known different fat contents, and the correspondence between the characteristic parameter to be measured and the fat contents is established as follows.

The phantoms with known different fat contents are mixed with a same proton density fat fraction (PDFF) phantom, reconstruction data of a mixed signal is acquired based on a corresponding RF pulse sequence, and a corresponding characteristic parameter is obtained.

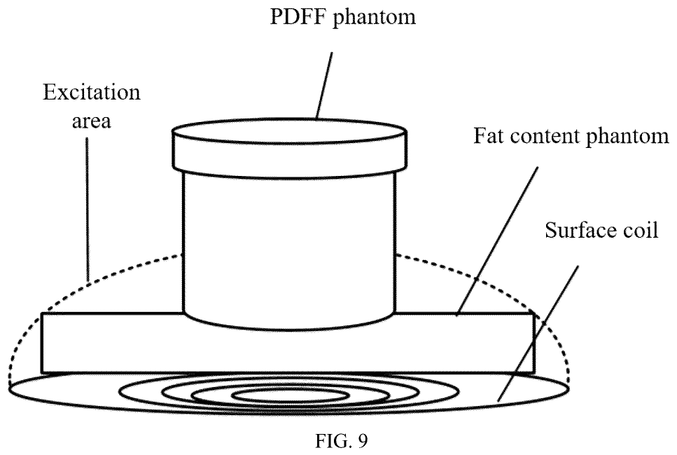

As shown in FIG. 9, phantoms with different fat contents and the same PDFF phantom are placed on the surface coil. For example, a phantom with a fat content of 10% is placed on the surface coil together with the PDFF phantom, or a phantom with a fat content of 15% is placed on the surface coil together with the PDFF phantom, etc. Then the MR signal in the excitation area of the surface coil includes a mixed signal of the fat content phantom and the PDFF phantom. The excitation area of the surface coil is fixed, and the mixed signal of the phantoms with different fat contents and the same PDFF phantom will result in different corresponding characteristic parameters of MR.

The correspondence between the characteristic parameter and the regional body fat content is established based on the reconstruction data of the mixed signal of a preset sample size and the corresponding characteristic parameter.

That is, through a series of phantoms with known fat contents, corresponding characteristic parameters are obtained by corresponding sequences and reconstruction methods (for example, characteristic parameter T2 is obtained by processing with the signal reconstruction data acquired by the CPMG sequence). Different fat contents correspond to different characteristic parameter values, such that the correspondence between fat content and characteristic parameters can be established. Therefore, the corresponding regional body fat content can be obtained according to the correspondence established by measuring the characteristic parameters.
Step S2. When the object to be measured is regarded as a mixed-substance object including a fat component and a non-fat component, an MR signal of the object to be measured is acquired by a corresponding RF pulse sequence, where the MR signal includes an MR signal of the fat component and an MR signal of the non-fat component. Undetermined coefficients of the fat component and the non-fat component in the MR signal are respectively determined by a multi-parameter fitting-based numerical calculation method. A fat content of the object to be measured is calculated according to the undetermined coefficients.

It should be noted that the object to be measured may be a mixed-substance object including a plurality of substances. For example, due to different body shapes and different thicknesses of subcutaneous fat, the object obtained based on the same sampling depth may include a single substance, such as 100% fat, or may include a plurality of mixed substances, such as fat, water and viscera. Therefore, when the object to be measured is a mixed-substance object, the fat fraction of the mixed substances can be estimated by multi-parameter fitting, and then the fat content of the body surface can be directly obtained. Preferably, when the object to be measured is regarded as a mixed-substance object, the MR signal of the object to be measured is acquired by a VTR-CPMG sequence, a CPMG sequence, or a SE-Diffusion sequence.

In a specific implementation of the step S2, when the MR signal of the object to be measured is acquired by the VTR-CPMG sequence, the undetermined coefficients of the fat component and the non-fat component in the MR signal are determined by the multi-parameter fitting-based numerical calculation method respectively, and the fat content of the object to be measured is calculated according to the undetermined coefficients, including:
establishing an MR signal calculation equation as follows:

$$min \{ ‖ {S}_{1} - {B}_{1} ( {a}_{1} - {b}_{1} {e}^{( - T R / T {1}_{1} )} {e}^{( - T E / T 2 )} ) - {B}_{2} ( {a}_{2} - {b}_{2} {e}^{( - T R / T {1}_{2} )} {e}^{( - T E / T 2 )} ) ‖ \} \qquad ( 7 )$$

where, S₁ is an intensity of an acquired VTR-CPMG echo signal; B₁ is a total signal intensity of a substance component of the non-fat component; and B₂ is a total signal intensity of a substance component of the fat component.

The undetermined coefficients B₁ and B₂ are determined by the multi-parameter fitting-based numerical calculation method, and a PDFF of the object to be measured is calculated according to the undetermined coefficients as follows:

$$PDFF = \frac{{B}_{2}}{{B}_{1} + {B}_{2}} \times 100 \% \qquad ( 8 )$$

In another specific implementation of the step S2, when the MR signal of the object to be measured is acquired by the CPMG sequence, the undetermined coefficients of the fat component and the non-fat component in the MR signal are determined by the multi-parameter fitting-based numerical calculation method respectively, and the fat content of the object to be measured is calculated according to the undetermined coefficients, including:
establishing an MR signal calculation equation as follows:

$$min \{ ‖ {S}_{2} - {B}_{1} {e}^{- t 2 / T {2}_{1}} - {B}_{2} {e}^{- t 2 / T {2}_{2}} ‖ \} \qquad ( 9 )$$

where, S₂ is an intensity of an acquired CPMG echo signal; B₁ is a total signal intensity of a substance component of the non-fat component; and B₂ is a total signal intensity of a substance component of the fat component.

The undetermined coefficients B₁ and B₂ are determined by the multi-parameter fitting-based numerical calculation method, and a PDFF of the object to be measured is calculated according to the undetermined coefficients as follows:

$$PDFF = \frac{{B}_{2}}{{B}_{1} + {B}_{2}} \times 100 \% \qquad ( 10 )$$

In another specific implementation of the step S2, when the MR signal of the object to be measured is acquired by the SE-Diffusion sequence, the undetermined coefficients of the fat component and the non-fat component in the MR signal are determined by the multi-parameter fitting-based numerical calculation method respectively, and the fat content of the object to be measured is calculated according to the undetermined coefficients, including:
establishing an MR signal calculation equation as follows:

$$min \{ ‖ {S}_{3} - {B}_{1} {e}^{- \frac{- {γ}^{2} {G}^{2} {tEE}^{3} D 1}{12} - \frac{tEE}{{T}_{2}}} - {B}_{2} {e}^{- \frac{- {γ}^{2} {G}^{2} {tEE}^{3} {D}_{2}}{12} - \frac{tEE}{T 2}} ‖ \} \qquad ( 11 )$$

The undetermined coefficients B₁ and B₂ are determined by the multi-parameter fitting-based numerical calculation method, and a PDFF of the object to be measured is calculated according to the undetermined coefficients as follows:

$$PDFF = \frac{{B}_{2}}{{B}_{1} + {B}_{2}} \times 100 \% \qquad ( 12 )$$

Based on the above disclosed content, in the embodiment of the present disclosure, when the object to be measured is regarded as a single-substance object, the MR signal of the object to be measured is acquired by a corresponding RF pulse sequence according to a characteristic parameter to be measured. The acquired MR signal is processed, and a target characteristic parameter value corresponding to the object to be measured is obtained. The characteristic parameter is calibrated by phantoms with known different fat contents, and a correspondence between the characteristic parameter to be measured and the fat contents is established. A fat content corresponding to the target characteristic parameter value is determined according to the correspondence, where the characteristic parameter at least includes a longitudinal relaxation time T1, a transverse relaxation time T2, and/or an ADC D. When the object to be measured is regarded as a mixed-substance object including a fat component and a non-fat component, the MR signal of the object to be measured is acquired by a corresponding RF pulse sequence. Undetermined coefficients of the fat component and the non-fat component in the MR signal are respectively determined by a multi-parameter fitting-based numerical calculation method. The fat content of the object to be measured is calculated according to the undetermined coefficients. Therefore, the present disclosure provides a calibration method for the fat content of a single-substance object, and provides a parameter fitting calculation method for the fat content of a mixed-substance object. The measurement results are accurate, providing solid data support for the testing of the MR system.

In a third aspect, the present disclosure provides a computer device, including a memory, a processor, and a transceiver which are sequentially connected in communication. The memory is configured to store a computer program. The transceiver is configured to send and receive messages. The processor is configured to read the computer program and execute the method described in any possible design of the first aspect.

For example, the memory may include, but is not limited to, a random access memory (RAM), a read-only memory (ROM), a flash memory, a first-input first-output memory (FIFO) and/or a first-input last-output memory (FILO), etc.; the processor may not be limited to a microprocessor of the STM32F105 series; the transceiver may be, but is not limited to, a WiFi (Wireless Fidelity) wireless transceiver, a Bluetooth wireless transceiver, a GPRS (General Packet Radio Service) wireless transceiver and/or a ZigBee (ZigBee protocol, a low-power local area network protocol based on the IEEE802.15.4 standard) wireless transceiver, etc. In addition, the computer device may also include, but is not limited to, a power module, a display screen, and other necessary components.

The working process, working details, and technical effects of the aforementioned computer device provided in the third aspect of this embodiment may refer to the method described in the first aspect or any possible design of the first aspect, and will not be repeated here.

In a fourth aspect, the present disclosure provides a computer-readable storage medium. The computer-readable storage medium is configured to store an instruction, and the instruction is run on a computer to execute the method described in any possible design of the first aspect.

The computer-readable storage medium refers to a carrier for storing data, which may include, but is not limited to, a floppy disk, an optical disk, a hard disk, a flash memory, a USB flash drive, and/or a memory stick, etc., and the computer may be a general-purpose computer, a special-purpose computer, a computer network, or other programmable devices.

The working process, working details, and technical effects of the aforementioned computer-readable storage medium provided in the fourth aspect of this embodiment may refer to the method described in the first aspect or any possible design of the first aspect, and will not be repeated here.

In a fifth aspect, the present disclosure provides a computer program product including an instruction. When the instruction is run on a computer, the computer executes the method described in any possible design of the first aspect.

The working process, working details and technical effects of the aforementioned computer program product containing instructions provided in the fifth aspect of this embodiment can be referred to the method described in the first aspect or any possible design in the first aspect, and will not be repeated here.

Finally, it should be noted that the above description is only a preferred embodiment of the present disclosure and is not intended to limit the scope of protection of the present disclosure. Any modifications, equivalent substitutions, improvements, etc. made within the spirit and principles of the present disclosure shall be included in the scope of protection of the present disclosure.

1. A magnetic resonance (MR) system for measuring a regional body fat content, comprising: a data processing subsystem, a radio frequency (RF) subsystem, and a magnet device, wherein
the RF subsystem comprises a spectrum analyzer, a power amplifier, a preamplifier, a transmit-receive (T/R) switch, and a surface coil module; the surface coil module comprises at least one set of surface coils; and a depth of an excitation area of the at least one set of surface coils is adapted to a subcutaneous depth.

2. The MR system according to claim 1, wherein the surface coil module comprises a set of TR integrated surface coils; and a depth of an excitation area and a depth of a receiving area of the set of TR integrated surface coils are both adapted to the subcutaneous depth.

3. The MR system according to claim 1, wherein the surface coil module comprises a set of surface excitation coils and a set of surface receiving coils; and a depth of an excitation area of the surface excitation coils is adapted to the subcutaneous depth.

4. The MR system according to claim 3, wherein the set of surface excitation coils comprises a plurality of surface excitation coils.

5. A method for measuring a regional body fat content using the MR system according to claim 1, comprising following steps:
when an object to be measured is regarded as a single-substance object: acquiring, by a corresponding RF pulse sequence, an MR signal of the object to be measured according to a characteristic parameter to be measured; processing the MR signal, and obtaining a target characteristic parameter value corresponding to the object to be measured; calibrating, by phantoms with known different fat contents, the characteristic parameter, and establishing a correspondence between the characteristic parameter to be measured and the fat contents; and
determining a fat content corresponding to the target characteristic parameter value according to the correspondence, wherein the characteristic parameter at least comprises a longitudinal relaxation time T1, a transverse relaxation time T2, and/or an apparent diffusion coefficient (ADC) D; and
when the object to be measured is regarded as a mixed-substance object comprising a fat component and a non-fat component: acquiring, by a corresponding RF pulse sequence, an MR signal of the object to be measured, wherein the MR signal comprises an MR signal of the fat component and an MR signal of the non-fat component; determining, by a multi-parameter fitting-based numerical calculation method, undetermined coefficients of the fat component and the non-fat component in the MR signal, respectively; and calculating a fat content of the object to be measured according to the undetermined coefficients.

6. The method according to claim 5, comprising: when the object to be measured is regarded as the single-substance object:
when the characteristic parameter to be measured is the longitudinal relaxation time T1, acquiring, by a varied-repetition time-Carr-Purcell-Meiboom-Gill (VTR-CPMG) sequence, the MR signal of the object to be measured;
when the characteristic parameter to be measured is the transverse relaxation time T2, acquiring, by a Carr-Purcell-Meiboom-Gill (CPMG) sequence, the MR signal of the object to be measured; and
when the characteristic parameter to be measured is the ADC D, acquiring, by a spin echo (SE)-Diffusion sequence, the MR signal of the object to be measured.

7. The method according to claim 6, wherein the step of processing the MR signal, and obtaining the target characteristic parameter value corresponding to the object to be measured comprises:
averaging MR signals acquired by the VTR-CPMG sequence based on a series of different repetition times (TR), and obtaining a signal intensity value A1 corresponding to different recovery times of a longitudinal magnetization vector as follows:
$$A 1 = {A}_{01} ( a - b {e}^{( - T R / T 1 )} {e}^{( - T E / T 2 )} ) \qquad ( 1 )$$
wherein A₀₁ is a signal intensity corresponding to a maximum longitudinal magnetization vector; a and b are function parameters configured to describe a recovery value of the longitudinal magnetization vector along a direction of a main magnetic field B₀, and in a case of ideal saturation-recovery, a=b=1; e is a natural logarithm; and TE is a fixed echo time (TE); and
calculating the longitudinal relaxation time T1 based on a minimum function min constraint as follows:
$$min \{ ‖ {S}_{1} - {\sum }_{i = 1}^{N} A {1}_{i} ( {a}_{i} - {b}_{i} {e}^{( - T R / T {1}_{i} )} {e}^{( - T E / T 2 )} ) ‖ \} \qquad ( 2 )$$
wherein ∥ ∥ is a 2-norm of the vector; S₁ is an intensity of a VTR-CPMG echo signal; N is a total number of substances in the object to be measured for which the longitudinal relaxation time T1 needs to be estimated; A1ᵢ is a total signal intensity of an i^{th} substance; aᵢ and bᵢ are function parameters configured for the i^{th} substance to describe the recovery value of the longitudinal magnetization vector along the direction of the main magnetic field B₀; and T1ᵢ is the longitudinal relaxation time T1 of the i^{th} substance.

8. The method according to claim 6, wherein the step of processing the MR signal, and obtaining the target characteristic parameter value corresponding to the object to be measured comprises:
averaging CPMG signals with same echo spacing acquired a plurality of times based on a fixed TE, and obtaining a decay signal intensity value A₂ of a transverse magnetization vector based on the fixed TE as follows:
$$A 2 = {A}_{02} {e}^{( - t 1 l {T}_{2} )} \qquad ( 3 )$$
wherein A₀₂ is a signal intensity corresponding to a maximum transverse magnetization vector; e is a natural logarithm; and t1 is a TE; and
subjecting CPMG echo signals to single-exponential fitting, and calculating, by a minimum function min constraint, the transverse relaxation time T2 as follows:
$$min \{ ‖ {S}_{2} - {\sum }_{i = 1}^{N} A {2}_{i} {e}^{- t 2 / T {2}_{i} )} ‖ \} \qquad ( 4 )$$
wherein S₂ is an intensity of the CPMG echo signal; A2ᵢ is a signal amplitude of an i^{th} substance; N is a total number of substances in the object to be measured; t2 is a time calculated from a maximum transverse magnetization intensity; and T₂ᵢ is the transverse relaxation time T2 of the i^{th} substance.

9. The method according to claim 6, wherein the step of processing the MR signal, and obtaining the target characteristic parameter value corresponding to the object to be measured comprises:
performing phase correction and accumulation on an acquired SE-Diffusion signal, and obtaining a correspondence between a signal intensity and a diffusion encoding time as follows:
$${S}_{3} = A 3 {e}^{- \frac{- {γ}^{2} {G}^{2} t E {E}^{3} D}{12} - \frac{tEE}{T 2}} \qquad ( 5 )$$
wherein A3 is an amplitude of the SE-Diffusion signal; γ is a magnetogyric ratio; G is a strength of a gradient magnetic field; and tEE is the diffusion encoding time; and
changing the diffusion encoding time tEE, averaging a series of acquired SE-Diffusion signals with different degrees of diffusion weighting, and calculating, by a minimum function min constraint, the ADC D as follows:
$$min \{ ‖ {S}_{3} - {\sum }_{i = 1}^{N} A {3}_{i} {e}^{- \frac{- {γ}^{2} {G}^{2} t E {E}^{3} Di}{12} - \frac{tEE}{T 2}} ‖ \} \qquad ( 6 )$$
wherein S₃ is an intensity of the SE-Diffusion signal; N is a total number of substances in the object to be measured for which the apparent diffusion coefficient D needs to be estimated; and A3ᵢ is a total signal intensity of an i^{th} substance.

10. The method according to claim 5, wherein the step of calibrating, by the phantoms with known different fat contents, the characteristic parameter, and establishing the correspondence between the characteristic parameter to be measured and the fat contents comprises:
mixing the phantoms with known different fat contents with a same proton density fat fraction (PDFF) phantom, acquiring reconstruction data of a mixed signal based on a corresponding RF pulse sequence, and obtaining a corresponding characteristic parameter; and
establishing the correspondence between the characteristic parameter and the regional body fat content based on the reconstruction data of the mixed signal of a preset sample size and the corresponding characteristic parameter.

11. The method according to claim 5, comprising: when the object to be measured is regarded as the mixed-substance object: acquiring, by a VTR-CPMG sequence, a CPMG sequence, or a SE-Diffusion sequence, the MR signal of the object to be measured.

12. The method according to claim 11, wherein the step of, when acquiring, by the VTR-CPMG sequence, the MR signal of the object to be measured, determining, by the multi-parameter fitting-based numerical calculation method, the undetermined coefficients of the fat component and the non-fat component in the MR signal, respectively; and calculating the fat content of the object to be measured according to the undetermined coefficients comprises:
establishing an MR signal calculation equation as follows:
$$min \{ ‖ {S}_{1} - {B}_{1} ( {a}_{1} - {b}_{1} {e}^{( - T R / T {1}_{1} )} {e}^{( - T E / T 2 )} ) - \qquad ( 7 )$$
$${B}_{2} ( {a}_{2} - {b}_{2} {e}^{( - T R / T {1}_{2} )} {e}^{( - T E / T 2 )} ) ‖ \}$$
wherein S₁ is an intensity of an acquired VTR-CPMG echo signal; B₁ is a total signal intensity of a substance component of the non-fat component; and B₂ is a total signal intensity of a substance component of the fat component; and
determining, by the multi-parameter fitting-based numerical calculation method, the undetermined coefficients B₁ and B₂; and calculating a PDFF of the object to be measured according to the undetermined coefficients as follows:
$$PDFF = \frac{{B}_{2}}{{B}_{1} + {B}_{2}} \times 100 \% . \qquad ( 8 )$$

13. The method according to claim 11, wherein the step of, when acquiring, by the CPMG sequence, the MR signal of the object to be measured, determining, by the multi-parameter fitting-based numerical calculation method, the undetermined coefficients of the fat component and the non-fat component in the MR signal, respectively; and calculating the fat content of the object to be measured according to the undetermined coefficients comprises:
establishing an MR signal calculation equation as follows:
$$min \{ ‖ {S}_{2} - {B}_{1} {e}^{- t 2 / T {2}_{1}} - {B}_{2} {e}^{- t 2 / T {2}_{2}} ‖ \} \qquad ( 9 )$$
wherein S₂ is an intensity of an acquired CPMG echo signal; B₁ is a total signal intensity of a substance component of the non-fat component; and B₂ is a total signal intensity of a substance component of the fat component; and
determining, by the multi-parameter fitting-based numerical calculation method, the undetermined coefficients B₁ and B₂; and calculating a PDFF of the object to be measured according to the undetermined coefficients as follows:
$$PDFF = \frac{{B}_{2}}{{B}_{1} + {B}_{2}} \times 100 \% . \qquad ( 10 )$$

14. The method according to claim 11, wherein the step of, when acquiring, by the SE-Diffusion sequence, the MR signal of the object to be measured, determining, by the multi-parameter fitting-based numerical calculation method, the undetermined coefficients of the fat component and the non-fat component in the MR signal, respectively; and calculating the fat content of the object to be measured according to the undetermined coefficients comprises:
establishing an MR signal calculation equation as follows:
$$min \{ ‖ {S}_{3} - {B}_{1} {e}^{- \frac{- {γ}^{2} {G}^{2} {tEE}^{3} D 1}{12} - \frac{tEE}{T 2}} - {B}_{2} {e}^{- \frac{- {γ}^{2} {G}^{2} {tEE}^{3} D 2}{12} - \frac{tEE}{T 2}} ‖ \} \qquad ( 11 )$$
determining, by the multi-parameter fitting-based numerical calculation method, the undetermined coefficients B₁ and B₂; and calculating a PDFF of the object to be measured according to the undetermined coefficients as follows:
$$PDFF = \frac{{B}_{2}}{{B}_{1} + {B}_{2}} \times 1 \; 00 \% . \qquad ( 12 )$$

15. The method according to claim 5, wherein each time when the characteristic parameter is calibrated by the phantoms with known different fat contents, phantoms with different fat contents and a same PDFF phantom are placed on the surface coil module of the MR system.

16. The method according to claim 5, wherein in the MR system, the surface coil module comprises a set of TR integrated surface coils; and a depth of an excitation area and a depth of a receiving area of the set of TR integrated surface coils are both adapted to the subcutaneous depth.

17. The method according to claim 5, wherein in the MR system, the surface coil module comprises a set of surface excitation coils and a set of surface receiving coils; and a depth of an excitation area of the surface excitation coils is adapted to the subcutaneous depth.

18. The method according to claim 17, wherein in the MR system, the set of surface excitation coils comprises a plurality of surface excitation coils.

19. The method according to claim 6, wherein the step of calibrating, by the phantoms with known different fat contents, the characteristic parameter, and establishing the correspondence between the characteristic parameter to be measured and the fat contents comprises:
mixing the phantoms with known different fat contents with a same proton density fat fraction (PDFF) phantom, acquiring reconstruction data of a mixed signal based on a corresponding RF pulse sequence, and obtaining a corresponding characteristic parameter; and
establishing the correspondence between the characteristic parameter and the regional body fat content based on the reconstruction data of the mixed signal of a preset sample size and the corresponding characteristic parameter.

20. The method according to claim 7, wherein the step of calibrating, by the phantoms with known different fat contents, the characteristic parameter, and establishing the correspondence between the characteristic parameter to be measured and the fat contents comprises:
mixing the phantoms with known different fat contents with a same proton density fat fraction (PDFF) phantom, acquiring reconstruction data of a mixed signal based on a corresponding RF pulse sequence, and obtaining a corresponding characteristic parameter; and
establishing the correspondence between the characteristic parameter and the regional body fat content based on the reconstruction data of the mixed signal of a preset sample size and the corresponding characteristic parameter.
